(19) United States
(12) Patent Application Publication
(10) Pub. No.: US 2025/0049292 A1
(43) Pub. Date: Feb. 13, 2025

(54) METHOD FOR AUTOMATED LOCALIZATION OF A SPECIFIC TISSUE TYPE DURING AN ENDOSCOPIC PROCEDURE, AND ASSOCIATED IMAGE RECORDING SYSTEM

(71) Applicant: Schölly Fiberoptic GmbH, Denzlinen (DE)

(72) Inventors: Michael Schwarzle, Denzlingen (DE); Markus Gern, Emmendingen (DE); Christoph Rapp, Freiburg (DE); Markus Debatin, Freiburg (DE); Pascal Striebel, Denzlingen (DE)

(73) Assignee: Schölly Fiberoptic GmbH, Denzlinen (DE)

(21) Appl. No.: 18/795,324

(22) Filed: Aug. 6, 2024

(30) Foreign Application Priority Data
Aug. 7, 2023 (DE) 102023120892.6

(51) Int. Cl. A61B 1/00; G06V 10/22; G06V 10/56

(52) U.S. Cl. CPC A61B 1/000094; A61B 1/000096; G06V 10/22; G06V 10/56; G06V 2201/03

(57) ABSTRACT

An approach for allowing the automated localization, within a recorded image (3), of a specific tissue type (6) within an operating region (9) using an image recording system (1). Here, provision is made for two temporally successive selection steps A and B to be used to identify and select those image regions (4) within the image which show the desired tissue type. The scope of the first selection step A includes both a spatial restriction of the image regions (5) and optionally a reduction in the spectral information to be processed and/or in the pixels to be processed of the respective image. Only in the second selection step B is the target tissue type actually to be determined then finally identified within the image and selected as second image regions so that a user can ultimately locate this tissue type within the region observed using the image recording system.

### CROSS-REFERENCE TO RELATED APPLICATIONS

This application claims priority from German Patent Application No. 10 2023 120 892.6, filed Aug. 7, 2023, which is incorporated herein by reference as if fully set forth.

### TECHNICAL FIELD

The invention relates to a method for automated localization of a specific target tissue type within at least one image recorded using an (in particular medical) image recording system. For example, this may occur during an endoscopic procedure performed using an endoscope as part of the image recording system. In this context, the method according to the invention should facilitate or even for the first time allow a very reliable distinction to be made between the target tissue type and other types of tissue within the image, and hence facilitate or allow localization of this tissue within an operating region (observed using the image recording system at that time).

### BACKGROUND

The prior art has already disclosed the practice of rendering visible specific tissue types to be identified, for example tumors, with the aid of fluorescent dyes such that the cancerous tissue can be identified for the surgeon in a live video image by way of appropriate colored marking. Moreover, surgeons also exploit the fact that the mechanical firmness of cancerous tissue differs from healthy tissue in order to find and localize the cancerous tissue by touch. However, in such approaches, the physician is often made to rely on their experience and anatomical knowledge in order to localize a specific tissue type within an operating region using an endoscope.

Nerve tissue is included among the critical structures in the human body, wherein injury to nerves during surgery can lead to different outcomes, including chronic pain or even the loss of function of various organic structures. This is often accompanied by a massive reduction in quality of life for the affected party.

The careful identification of nerves to avoid nerve damage is imperative, especially in the case of surgery in the head and neck region. However, the surgeon currently identifies the nerves only on the basis of anatomy and appearance, optionally in combination with electromyography (EMG), with nerve damage often actually being determined after the surgery has been completed. In the meantime, scientific research proposing optical methods such as diffuse reflectance spectroscopy (DRS) and fluorescence spectroscopy (FS) to identify nerves in targeted fashion has already been published, for instance the article “In Vivo Nerve Identification in Head and Neck Surgery Using Diffuse Reflectance Spectroscopy” by Langhout et al., published in 2018 in the journal “Laryngoscope Investigative Otolaryngology”.

Thus, there currently are already various state-of-the-art technological approaches for localizing peripheral nerves: One of these approaches is the method of autofluorescence (AF), which makes use of the fact that different tissue types exhibit fluorescence of different strengths when excited at suitable wavelengths. In this case, the autofluorescence method is based on the fact that natural fluorophores (for example fat pigments, collagen or elastin) are present in the human body and are excitable to perform autofluorescence in the case of appropriate illumination using UV light. However, this method therefore does not only render solely nerves visible, but also other tissue types that exhibit autofluorescence properties, for example ligaments or adipose tissue. However, in medical application, the nerve must be clearly distinguished from these other tissue types in order to prevent said nerve from being injured during the operation. The decisive disadvantage of the autofluorescence method therefore lies in the fact that not only nerve tissue but other tissue types as well exhibit autofluorescence properties, and so these other tissue types become also visible in autofluorescence imaging. The described autofluorescence method is therefore not specific enough in order to be able to exclusively and selectively detect and localize nerve tissue.

Then again, physicians frequently use the visual impression obtained, experience and anatomical knowledge within the scope of the endoscopic localization of nerves. However, these impressions are all subjective, and the anatomy in particular can exhibit differences from patient to patient. For these reasons, too, various technologies are being trialed in order to allow nerve tissue to be localized more reliably and more reproducibly during an endoscopic procedure and hence avoid injury to nerves during the procedure.

A further possible method which can be used for nerve detection is, as mentioned, the method of diffuse reflectance spectroscopy (DRS). In this method, reflectance spectra with a high wavelength resolution of for example 1 nm are typically used to distinguish different tissue types from one another on the basis of their “spectral fingerprint”. However, the corresponding measurement probe is directly placed onto, or even pierced into, the tissue in the process. Thus, for DRS, a probe must be in direct contact with the tissue and then accordingly also only supplies information for a specific location in the current field of view. However, this is impractical for endoscopic applications, especially whenever a relatively large operating region must be examined. A localization of specific tissue types within the entire field of view of an endoscope is precisely not possible using this method.

### SUMMARY

Against this background as a starting point, the problem addressed by the invention is that of making available an improved technical approach that allows localization of a specific tissue type, in particular nerve tissue, within an operating region observed at that time using an endoscope for example.

To solve this problem, the one or more of the features disclosed herein are provided in a method according to the invention. In particular, to solve the problem the invention therefore proposes within the scope of a method of the type set forth at the outset the practice of in a first selection step identifying and selecting at least one first image region within the at least one image in automated fashion using a first imaging method. In this context, the at least first image region also visualizes at least one other secondary tissue type that deviates from the target tissue type in addition to the target tissue type. In other words, this first preselection is not yet sufficiently selective to localize the target tissue type sufficiently accurately in the image. However, the result of the first selection step can already be an image with a reduced quantity of information.

Therefore, according to the invention, at least one second image region (as a portion) is identified and selected within the at least one already preselected first image region in a second selection step by means of a second imaging method that deviates from the first imaging method (just like the first imaging method as well can preferably be an optical method in each case). In this case, the at least one second image region then visualizes predominantly or even exclusively the target tissue type to be determined. In other words, the target tissue type can consequently be localized sufficiently selectively within the operating region on the basis of the second image region. Thus, the result of the second selection step can be an image with an even further reduced quantity of information. Hence, in particular, the image obtained thus can be a spectral image having the desired information, specifically the relative position and extent of the target tissue type within the original at least one image.

Hence the method can comprise two temporally successive image processing steps, specifically the first and the second selection step.

Especially in a third, subsequent method step, the at least one second image region identified thus can then be highlighted for a user in the at least one image or in an image corresponding thereto, for instance a VIS image (for example, this can be a freeze frame or, for example, a changing live image). This can be implemented by way of specific false colors or, for example, with the aid of a so-called “image overlay”.

Using this method, it is possible to make a statement with pixel accuracy and in automated fashion (without any input by the user) as to whether or not the respective pixel shows the target tissue type. It may hold true here in the specific application that the at least one second image region has a lower resolution than the underlying image (which may be a VIS or IR live image, for example) observed by the surgeon and/or currently recorded by the image recording system.

In principle, the method according to the invention can further be used both in open medical procedures and in endoscopic procedures occurring in the interior of the body. The image recording system used in the method may for example comprise an endoscope, a microscope or a macroscope since the manner in which the image data are optically captured is not important.

Thus, the method according to the invention renders it possible to identify a specific tissue type within an image recorded using the image recording system, in particular within a live video frame data stream, this identification being quick (in particular in real time on the basis of a live video frame data stream), very reliable and spatially resolved. The main use of the application in this case lies in an improved way of conducting an operation: In particular, injury to nerve tissue can thus be prevented, whereby complications can be effectively avoided. The method can moreover assist the physician with their decision making in specific situations during an operation.

Thus, the surgeon can be assisted by the use of technology, especially when localizing peripheral nerves during an endoscopic operation. In this case, the method according to the invention takes the specific boundary conditions arising during endoscopic procedures into account, especially the necessity of contactlessly identifying the tissue type to be localized. The method enables a contactless and reliable localization of nerves in the entire current and changing field of view of an endoscope.

As described above, the invention provides for the combination of at least two different imaging methods (these can also be considered to be “measurement methods”) or measurement methods for identifying a specific type of tissue, for example nerve tissue, within a just recorded image and localizing said specific type of issue within the image. This approach is very advantageous, especially for the localization of nerves in the peripheral nervous system, as explained above.

For example, the first method step can initially use autofluorescence to classify in the entire image which first image regions show tissue types with a high autofluorescence activity. Subsequently, in the second method step, those second image regions which represent/contain the type of tissue to be identified (target tissue type) can then be identified and localized within the first image regions identified thus. For example, the following imaging methods can be used in this second method step: (a) fluorescence using extrinsically introduced fluorophores; (b) polarization analysis; (c) laser spectral contrast imaging (LSCI); and (d) the color ratios method.

The combination of different imaging technologies for localizing specific tissue types is a novel approach which in particular enables a specific distinction to be made between nerve tissue and other tissue types, as are observed in a typical operating situation, especially during an endoscopic procedure. It is advantageous, inter alia, that localization can be implemented in the entire field of view of the endoscope and that no tissue contact of any form is required to this end.

A second possible application for the invention lies in the localization and visualization of blood vessels. This is because injury to blood vessels is very bothersome in many operations as any bleeding must first be stopped before the operation can be continued. It can be particularly bothersome in this case that the endoscope optics are contaminated with blood. Such inadvertent injuries can also be avoided using the method according to the invention if the second selection step is designed with respect to the identification of blood vessels.

Specifically deviating from the DRS method mentioned at the outset, the method according to the invention can in this case provide, especially within the scope of the second method step, for only a small number of spectrally tightly delimited wavelength ranges (i.e. specific tightly delimited color ranges/colors) to be used/analyzed in order to distinguish between different tissue types. Thus, there is no need to record a complete continuous spectrum with a high resolution, as this generally is only possible by means of a complex spectrometer that is difficult to miniaturize. Thus, the second method step can be carried out without using a spectrometer in particular; this will still be explained in detail.

Since the method according to the invention can be carried out contactlessly, the used measurement optics, which can be integrated in the endoscope used in the method, need not be placed directly on the tissue; instead, these measurement optics can be kept at a distance from the tissue during the measurement, as is the case for typical endoscopic procedures. This is advantageous in that corresponding localization of nerves can be performed in the entire (movable) field of view of the endoscope/microscope/macroscope.

The target tissue type to be determined can be considered to be a primary tissue type which is at the center of attention. In this case, the second image region can already exclude, i.e. precisely not represent or only represent to a negligible extent, the at least one secondary tissue type (which may belong to a group of tissues to which the target tissue type also belongs). Further, the first image region may exclude at least one further tertiary tissue type present/visualized in the entire image, i.e. precisely not represent this or only represent this to a negligible extent. By contrast, both the primary and also at least one secondary tissue type can be visualized/present in the at least one first image region.

In typical application situations, for example arteries, veins, adipose tissue, muscles, nerve tissue, the liver or the intestine as different tissue types, and also objects foreign to the body, for instance metallic objects like a surgical instrument, may be visible/visualized in an image recorded with the image recording system. With the aid of the first (pre-) selection step, it is consequently possible to undertake spatial prefiltering within a just recorded image and thereby identify those image regions which show the target tissue type to be localized and possibly also further tissue types which from an optical point of view behave in a manner at least similar to the target tissue type to be localized, i.e. for example supply an autofluorescence signal like the latter. Consequently, the first image region can visualize for example a certain group of tissue types (for example, this can be a specific subgroup of specific tissue types). Further, the first image region can already exclude at least one tissue type present/visualized in the (overall) image (for example, a tissue type supplying no AF signal).

The described method can supply location information, specifically the at least one second image region identified in the recorded image (especially in a current live image), to a surgeon on the basis of the second selection step. This location information allows the surgeon to plan and/or conduct a subsequent therapeutic method step. However, the method according to the invention itself is not directed to the implementation of this therapeutic step, rather only to the provision of information for preparing the subsequent therapy and/or diagnosis, for example a specific tissue resection, which is optically observed using the endoscope and conducted using a separate operating tool. Hence, the method according to the invention can in particular provide for precisely no therapeutic or diagnostic step to be carried out, but only for information to be made available for the purpose of planning and preparing a subsequent therapeutic and/or diagnostic step.

In this case, the mentioned endoscope can be part of the image recording system, wherein the latter may for example also still comprise a monitor and/or a camera control unit.

The recorded image can be a single image (“still image”) or for example be part of a live video frame data stream, which is or was recorded continuously using the image recording system.

For example, the first measurement method can be imaging on the basis of diffuse reflectance spectroscopy (DRS) or by means of autofluorescence (AF), for example. In the latter AF method, fluorophores occurring naturally in human tissue are excited to emit light with the aid of (typically invisible) excitation light (e.g. in the UV wavelength range), said light emission then being able to be captured by sensors and typically being located at 500 nm. Following the evaluation of such an autofluorescence signal recorded with the aid of the image recording system, the first image regions selected thus can only still show adipose tissue and nerve tissue, for example. In this case, the excitation light can be excluded from imaging by means of an excitation light filter.

As mentioned, the method according to the invention can in the process in particular make use of so-called measured color ratios in order to differentiate between different tissue types. For example, such a color ratio can be understood here to mean a ratio of respective intensities of a picture element in relation to two different wavelength ranges (in particular with a respective bandwidth of for example in each case less than 200 nm, preferably less than 100 nm and/or at least 10 nm). To determine such wavelength ratios (synonyms include color ratios), respective measured intensity values from the individual different wavelength ranges (which may however partially overlap under certain circumstances) are initially required. Such spectrally selective intensity values can be obtained in two basic ways:
(i) Firstly, a wavelength sensitivity can be realized in the camera head of the system. According to this approach, use can be made of e.g. hyperspectral sensors, with there being different technologies available for the realization of such sensors. Inter alia, so-called “snapshot sensors” which provide only a few (e.g. 16) different color channels are also suitable for the approach according to the invention. This is because a very high spectral resolution is generally not required within the approach according to the invention. However, in an image recording system according to the invention, different wavelength ranges can also be captured for example by means of a beam splitter and a wavelength-selective (e.g. dichroic) layer; in this case, use can be made of conventional color sensors and/or monochromatic image sensors. A spatial separation between different wavelengths can be obtained in this way, with the result that two different spectral wavelength ranges can be steered to different (spatially separated) image sensors of the system and can be captured separately there by sensors.
(ii) Secondly, the wavelength sensitivity can also be based on a (temporally modulable) light source (in addition or as an alternative to the approach (i)), which is used together with the image recording system when imaging. For example, only a specific wavelength range can ever be output by the light source in a temporal sequence. Such a temporally varying spectral illumination can thus result in a temporal separation of different wavelength ranges, with the result that-even if only a single image sensor is used—different spectral wavelength ranges can be captured separately by sensors at different times.

All these approaches thus allow the ascertainment of the necessary intensity values in different wavelength ranges, which are required for the calculation of color ratios.

Unlike conventional approaches of hyperspectral imaging, it is precisely not necessary here to capture a complex color fingerprint of a specific tissue type with high spectral resolution, however. Instead, according to the invention, it is possible and more effective to initially empirically ascertain how specific color ratios are manifested in a specific tissue type. Subsequently, when the method according to the invention is carried out, the tissue type present can then be deduced directly and very much more reliably and quickly on the basis of such measured color ratios (and in particular on the basis of such empirical data), without this requiring a complicated broadband capture and evaluation of spectral measurement data (for instance, over the entire visible range+adjoining nonvisible wavelength ranges). In this case, the reduction in complexity is obtained by targeted selection of the respective spectral range which is taken into account when calculating the respective color ratio. This selection depends on the tissue type to be determined and may be ascertained by way of preliminary trials.

In the described method of using color ratios, the challenge thus lies in the identification of suitable spectral regions (as narrowband as possible) which should be used for distinguishing between the different tissue types, i.e. in the selection of the specific color ratios that should be calculated in the respective selection step. This is because these spectral regions must enable the localization of a specific tissue type in their totality without interindividual differences in the tissue (from patient to patient) or other interfering influences adversely affecting the localization in the process. However, since the differences between the numerous tissue types occurring in the human body are very large, it is a substantial challenge to empirically identify an appropriately sufficiently large number of suitable spectral ranges. However, this can be accomplished by preliminary trials and then taken into account when designing the image recording system.

In this context, too, the invention supplies a substantial advantage because the first method/selection step already reduces the solution space to such a great extent that the desired specific distinction between the tissue types can even only be realized with the aid of an optical method such as e.g. DRS and/or on the basis of measured color ratios in the second step. The spatial prefiltering implemented in the first selection step, for example on the basis of AF, already allows restriction of the image regions to be differentiated to such an extent that the DRS method or another imaging or measurement method (for example the aforementioned determination of color ratios)-even in the case of only a small spectral resolution—supplies usable results in the second selection step.

Accordingly, the problem can also be solved by further advantageous embodiments described below and in the claims.

Thus, the first selection step and/or the second selection step can be performed in automated fashion by the image recording system, in particular on the basis of an image evaluation of the at least one image. For example, an image evaluation can also be understood to mean a pixel-based calculation of color ratios for individual image regions.

The first and the second selection step can further be carried out continually in real time.

Further, the target tissue type can be visually highlighted, for example in a live video frame data stream. This allows a user to be able to localize the target tissue type particularly easily within a region observed using the image recording system.

Further, for a robust capture of the target tissue type, it is advantageous if the first selection step and/or the second selection step is carried out on the basis of partial spectral information which is spatially or temporally separated from overall spectral information relating to the at least one image. In other words, the image processing which forms the basis of the first and/or the second selection step is applied only to some of the information in relation to the overall spectral information supplied by the respective image. Thus, in particular, the at least one image for example can cover the entire visible wavelength spectrum (and thus be a VIS image) while the first selection step and/or the second selection step is applied to a spectral image which, although showing the same scene/the same image portion as the at least one VIS image (in which the target tissue type should ultimately be visualized for the user), only reproduces some of this overall spectral information or information deviating therefrom. Thus, for example, the first and/or the second selection step can be applied to an infrared image, in particular a fluorescence image whose spectral information is located outside of the visible range. Following the selection of the second image regions within this spectral image, said second image regions can then be visualized for the user in the VIS image.

The identification and selection of the second image regions with the aid of the second selection step is implemented particularly reliably and quickly in this case if the first selection step is used to reduce a quantity of image information (e.g. a quantity of image pixels to be processed) and/or a quantity of spectral information (e.g. a quantity of color channels to be evaluated/per pixel) processed in the second selection step. Thus, the first selection step A can be used to reduce the solution space of possible tissue types that have to be distinguished in step B. The fewer tissue types that need to be distinguished, the less information in the form of wavelength ranges is required to this end in the second selection step B. In practice, there are also cases in which the approach according to the invention only renders possible a distinction of tissue types in this manner (since the identification by means of a single selection step is often not possible with the hardware available). In the image recording system itself, this means that fewer wavelength ranges need to be recorded/captured by sensors. For example, this can result in the fact that a number of wavelength ranges which are created successively in time by means of a modulable light source and would be (actually) required for robust identification of the target tissue is reduced, or that for example a small number of color channels made available by a hyperspectral sensor of the system is already sufficient to reliably design the tissue identification. In other words, the approach according to the invention can significantly reduce the requirements in terms of system hardware, and hence open up new fields of application. Using the approach according to the invention, it may for example be sufficient to read out only 16 color channels/spectral bands of an image sensor, and not more than 100 different channels as in already known approaches that require a high spectral resolution. This also shows the specific technical advantages of the invention.

Considered from a different point of view, the method according to the invention can thus be designed such that only a limited spectral range is evaluated in the first selection step and/or in the second selection step. In this case, this limited spectral range can deviate from, in particular be smaller than or non-overlapping with, an (in particular broader) spectral range which is ultimately displayed to the user in the at least one image (e.g. the entire visible range).

A possible implementation of the method according to the invention provides for the scope of the second selection step to comprise the calculation of so-called color ratios on the basis of at least two different color channels of the image recording system for different picture elements in the at least one image. The respective color ratio in this case specifies an intensity ratio of at least two spectral ranges for the respective picture element or picture portion. In this case, it should be preferable for the respective spectral ranges on the basis of which the color ratios are determined to have a minimum width of at least 10 nm, preferably of at least 15 nm, or even of at least 20 nm. This distinguishes this approach from the use of conventional spectrometers for tissue analysis, in which the spectral resolution is typically a few nm or even below 1 nm. In this case, the lower spectral resolution while simultaneously identifying the target tissue type more reliably is rendered possible by the preselection which is supplied by the first selection step. For example, such an approach can already be realized using the three color channels of a conventional RGB sensor, which each capture spectral ranges of more than 50 nm, which also overlap under certain circumstances.

A preferred application of the method provides for the target tissue type to be nerve tissue and/or for the at least first image region to also visualize adipose tissue in addition to nerve tissue. In this case, the method is thus used to identify nerve and adipose tissue in the first selection step in order to subsequently differentiate the nerve tissue from the adipose tissue in the second selection step.

For example, provision can be made (especially for the just aforementioned purpose of differentiating nerve tissue) for the first imaging method to be imaging on the basis of autofluorescence while the second imaging method is based on diffuse reflectance spectroscopy. Within this approach, the UV excitation light used to excite the AF can be kept away from the image sensor of the image recording system with the aid of an excitation light filter.

Without using the first imaging method and the preselection resulting therefrom, it would be very difficult to identify and localize nerve tissue, for example, directly on the basis of measured reflected light using the DRS method: This is because, firstly, there are very many different tissue types which each show different typical colors that can be determined in the diffuse reflection. Moreover, however, the color of tissue may also vary from patient to patient; for example, perfusion may be better or not so good, or certain discolorations may occur, for example on account of metabolic disorders, especially a yellow coloration of specific tissue. All of this would make it very complicated to carry out the localization of the target tissue type directly using the DRS method. To this end, it would be necessary to resort to a spectrometer able to analyze the diffuse reflection spectrally with an accuracy of a few nanometers. However, the implementation thereof, especially in endoscopic applications, is very complicated to not at all possible because a spectrometer requires huge amounts of space.

For example, the presented approach can be implemented in an image processing algorithm that is deterministic and able to determine a specific tissue type, for example nerve tissue, quickly and efficiently and with high reliability on account of the pre-reduction of the solution space performed using the first measurement method.

A preferred variant in this case provides for the diffuse reflectance spectroscopy to be modified in such a way that a measurement is carried out without a spectrometer and/or contactlessly. For example, this can be implemented by virtue of a plurality of color ratios being calculated on the basis of a plurality of color channels (in particular one or more color image sensors) of the image recording system used during the method, based on respective measurement values ascertained in certain wavelength ranges (per pixel) using the image recording system. For example, if use is made of three beam splitters and in each case three color image sensors with three color channels, then it is possible to create nine color channels which can be used to calculate different color ratios.

For example, such an approach can be implemented using hardware as follows:

System 1: Broadband illumination, for example by means of a xenon lamp; use of a beam splitter in the image recording system such that different wavelength ranges can be captured using at least two image sensors. In this case, at least one of the two image sensors can be a color image sensor having a plurality of color channels. Using this approach, it is possible to form 3, 4, 5, 6 or even more different color channels. Hence, it is also possible to calculate a plurality of color ratios pixel-by-pixel by virtue of the signals from the respective color channels being appropriately combined with one another by calculation. In this context, four parameters should be defined for a specific color ratio: For example, if a ratio is formed from the wavelength range 470±5 nm and 620±10 nm, then four values should be specified for the respective start and end value of the respective wavelength interval (e.g.: 465, 475; 610, 630 in the example above).

In such an approach, the target tissue type is consequently identified on the basis of measured color ratios. In this context, this approach operates purely empirically: Unlike previously in the prior art, the DRS method should according to the invention precisely not be used to determine meaningful physiological parameters, for example a fat proportion or a proportion of beta-carotene or a ratio of oxygenated hemoglobin to deoxygenated hemoglobin (this ratio depends on the oxygen saturation of the blood). This is because the previously known approaches use spectrometers and DRS to determine such physiological parameters, and the tissue type is only deduced subsequently from such determined physiological parameters.

By contrast, the approach according to the invention proposes the direct determination of the target tissue type (without calculation of physiological parameters) from measured color ratios.

System 2: In the simplest case, an image recording system used within the approach according to the invention may comprise only one monochrome image sensor but use different illumination light sources, for example three different LEDs. Should the light sources be operated discontinuously, for example in pulsed fashion, they emit spectrally different illumination light at different times. This also makes it possible to in each case capture in spatially resolved fashion image signals in different spectral ranges, and these image signals can then be used to calculate color ratios. However, an RGB sensor or an HSI (hyperspectral imaging) sensor could also be used in such an approach according to the invention. Unlike in the use of (possibly single) broadband illumination, however, no beam splitter needs to be used here because the spectral split is implemented not spatially but temporally. Nevertheless, a beam splitter could also be used in this case, for instance in order to improve the spectral resolution by virtue of the captured illumination light for each LED being spectrally split in space again with the aid of the beam splitter and appropriate dichroic filters.

Moreover, the required spectral information that needs to be collected in order to determine the color ratios can also be ascertained with the aid of a scanning approach, in which spectral image information from a picture element is spatially distributed on an image sensor used in the image recording system, for instance using a scanning mirror or under time-varying illumination.

According to a further configuration of the method according to the invention explained above, provision can be made for the first imaging method to be imaging on the basis of autofluorescence while the second imaging method is likewise based on fluorescence, but on a fluorescence created by fluorophores introduced in the object region (observed using the image recording system); i.e., the fluorescence imaging in the second selection step is implemented here using at least one fluorophore which is introduced into a region observed using the image recording system. In this specific approach, the two fluorescence signals (that of the autofluorescence and the signal created by means of the at least one fluorophore introduced) can be distinguished because the AF is excited using UV light while, for example if ICG (indocyanine green) is used as a possible fluorophore, work is carried out with an excitation wavelength of 800 nm and the fluorescence signal is therefore not located at 500 nm, as in the case of autofluorescence, but at approximately 830 nm. In other words, the respective fluorescence signals evaluated in the first and second selection step, respectively, may thus differ spectrally. In this approach, a pre-selection can thus be made first by means of AF imaging, and the second image regions can subsequently be identified within the first image regions with the aid of the fluorophore-based imaging. This also allows a surgeon to perform a resection such that nerve tissue, for example, is not damaged.

However, a method according to the invention could also be realized in the reverse case, i.e. if the first selection step is initially conducted on the basis of fluorescence (i.e. on the basis of fluorophores extrinsically introduced into the body), and subsequently the at least one second image region is determined on the basis of a captured AF signal.

According to a further configuration of the method according to the invention explained above, provision can be made for the first imaging method to be imaging on the basis of autofluorescence, while the second imaging method is based on laser speckle imaging (LSI). In this case, the collective term “laser speckle imaging” can also be understood to mean, in particular, methods such as laser speckle contrast imaging (LSCI) and laser speckle contrast analysis (LASCA). Accordingly, the method can be characterized in such a case by work being conducted with coherent illumination in the second selection step in order to create the desired speckle patterns, while incoherent illumination is used in the first selection step. Thus, microscopic movements of the tissue, for example the expansion of blood vessels or their pulsation, can be captured on the basis of interference. In this case, it was found that nerve tissue often comprises well perfused connective tissue, which is traversed by very thin blood vessels, and so even nerve tissue can be selectively identified with the aid of LSI. In this specific approach, the differentiation of the target tissue type in the second selection step thus takes place with the aid of an interference motion analysis.

According to yet another configuration of the method according to the invention explained above, provision can be made for the first imaging method to be imaging on the basis of autofluorescence, while the second imaging method is based on polarization. Here, this specific approach is particularly suitable for determining nerve tissue because nerves are typically present in the form of fine nerve strands which all run in the same direction and hence are very ordered, and this can then be captured in automated fashion in an operating scene by means of polarization imaging and appropriate image evaluation.

In summary, it is thus recognized that the generic approach according to the invention provides for the first and the second measurement or imaging method to differ in each case and for one of (i) differing fluorescence signals and (ii) an optically captured motion signal (in the case of LSI) and (iii) information (optically captured) regarding a structure of the tissue (e.g. in the case of polarization) to be used for the further differentiation of tissue in the second selection step. What needs to be taken into account here is that no tissue-specific fluorophores (yet to be developed) need to be used; instead, it is possible to resort to conventional fluorophores or even possible to entirely manage without the use of (extrinsic) fluorophores.

Thus the invention solves the fundamental problem that the aforementioned measurement methods alone are often not specific enough to localize a specific tissue type, for instance nerve tissue, in an operation situation. What needs to be taken into account here is that especially endoscopic procedures have technical boundary conditions in respect of the spectral resolution which can be met using the respective image recording system. The 2-step approach of the invention now solves this problem by virtue of the solution space being reduced significantly in the first selection step and a differentiation and localization of the tissue type still being able to be reliably obtained in the second selection step even if only limited spectral information is available.

### BRIEF DESCRIPTION OF THE DRAWINGS

The invention will now be described in more detail on the basis of exemplary embodiments, but is not restricted to these exemplary embodiments. Further developments of the invention can be obtained from the following description of a preferred exemplary embodiment in conjunction with the general description, the claims, and the drawings.

In the following description of various preferred embodiments of the invention, elements that correspond in terms of their function are denoted by corresponding reference numerals, even in the case of a deviating design or shape.

In the figures:

FIG. 1 shows an example of an image recording system according to the invention, by means of which the method according to the invention can be applied,

FIG. 2 shows a schematic illustration of the essential method steps of the method according to the invention when temporally constant illumination is used and the individual spectral image information is spatially split,

FIG. 3 shows a further schematic flowchart which explains the method according to the invention,

FIG. 4 shows a further image recording system, albeit with time-modulated illumination,

FIG. 5 shows a possible configuration of the method according to the invention when using the image recording system shown in FIG. 4, and finally

FIG. 6 shows a further possible implementation of a method according to the invention using an HSI (hyperspectral imaging) camera.

### DETAILED DESCRIPTION

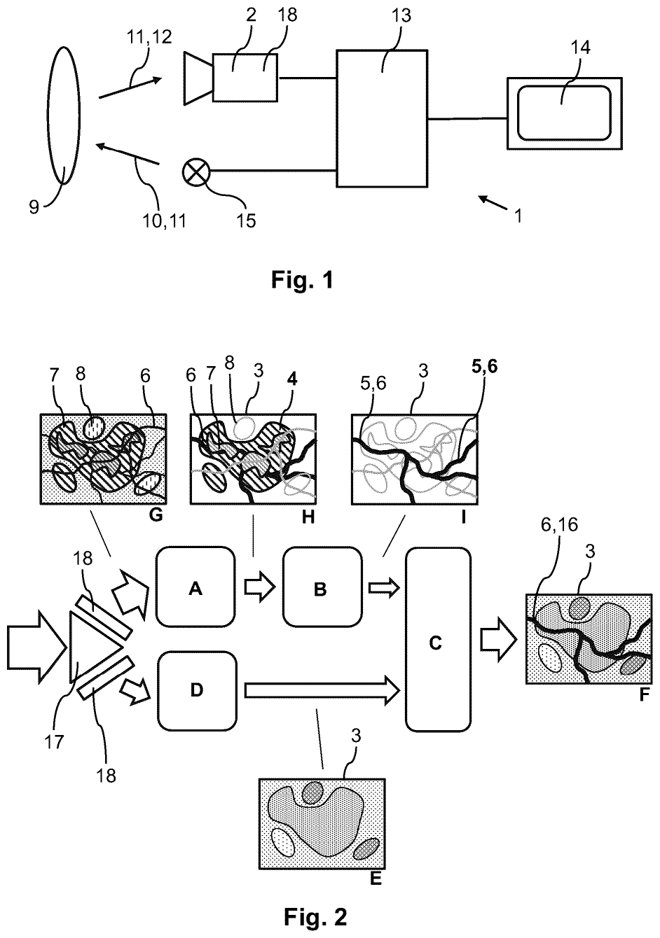

FIG. 1 shows an image recording system 1 which comprises an endoscope 2 having an image sensor 18 and an associated light source 15, the latter being able to be used to transmit both illumination light 11 and invisible UV excitation light 10 to a region 9 observed by the endoscope 2. In this case, the image sensor 18 of the endoscope 2 continually captures images 3 in the form of a video frame data stream, these images being processed by a downstream camera control unit 13 and subsequently being reproduced in the desired form on a monitor 14.

As identified schematically in FIG. 2, the endoscope 2 further comprises a beam splitter 17 with associated spectral optical filters that allow the overall spectral image information recorded by the optics of the endoscope 2 to be spatially split and assigned to a respective image sensor 18. The lower imaging path, denoted by the letter D, consequently provides for conventional image processing of the visible wavelengths in order to thus create the shown visible processed image D (VIS image). Additionally, the camera control unit 13 however also still implements the upper image processing path shown in FIG. 2; it comprises two image processing steps A and B. The image information, spectrally selective under certain circumstances and captured by way of the endoscope 2, more precisely the beam splitter and the upper image sensor 18, is depicted in the left upper image here. A secondary tissue type 7, specifically adipose tissue, and further tertiary tissue types 8 are also visible in the image in addition to nerves as the target tissue type 6 to be determined. The primary tissue type 6 to be localized and the secondary tissue type 7 in this case belong to the same tissue group and create an approximately comparable autofluorescence signal. Thus, if the light source 15 shown in FIG. 1 irradiates the region 9 using a suitable excitation wavelength in the UV range, then both of these tissue types 6 and 7 emit a corresponding autofluorescence signal, which then reaches the upper image processing chain in FIG. 2.

In order to now identify the desired target tissue type 6, specifically the neural pathways within the operating region 9, and highlight/visualize these for the surgeon so that said surgeon can localize or find the nerves in the operating region 9, the invention provides for, in the first selection step A, firstly the first image regions denoted by reference sign 4 to be automatically identified and selected by the camera control unit 13 in the current image 3 by means of autofluorescence imaging (and appropriate image processing of the images recorded thus), as illustrated on the basis of the highlighting in the top middle detail image H in FIG. 2. In this case, these first image regions 4 also comprise the deviating secondary tissue type 7 in addition to the target tissue type 6.

Only in a subsequent second selection step B are, likewise in automated fashion by the camera control unit 13, a plurality of second image regions 5 identified and selected within the first image region 4 with the aid of a second imaging method that deviates from the autofluorescence method. These second image regions 5 are highlighted in black in the top right detail image I in FIG. 2 and predominantly to exclusively only still comprise nerve tissue, i.e. the desired target tissue type 6.

As indicated in FIG. 2, the image information obtained in the lower image processing chain (the VIS image) can then be fused in a third method step C with the additional information obtained with the aid of the two selection steps A and B regarding the relative position of the target tissue type 6 within the image 3. As a result as shown in the detail image F, the user can thus be presented with a so-called overlay image F, in which the identified image regions 6, i.e. the nerves to be localized, are highlighted in the VIS image E as a superposition.

It should still be mentioned that work in the approach according to FIGS. 1 and 2 is carried out with time-constant illumination, wherein the partial spectral information, on the basis of which the first selection step A and the second selection step B are performed, is spatially separated off/separated from the overall spectral information (optically captured by the endoscope 2) with the aid of the beam splitter 17.

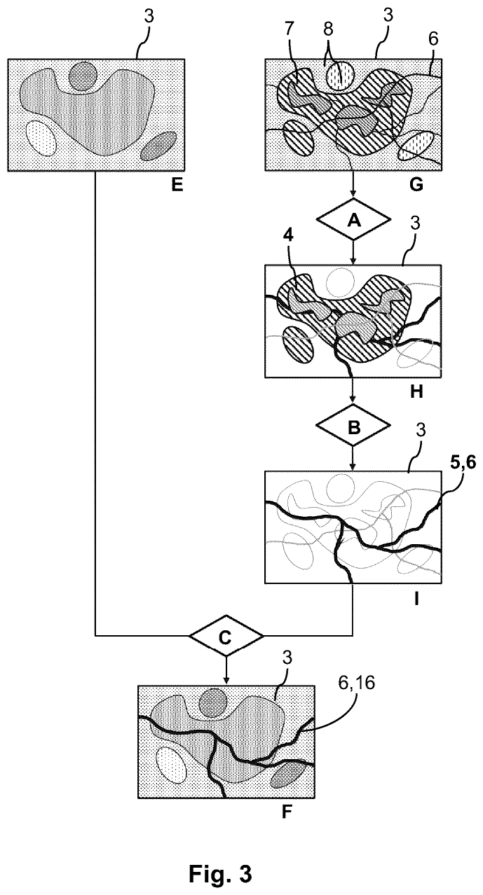

The aforementioned individual steps of the method according to the invention and the images created thereby are also illustrated again in the overview-like schematic illustration in FIG. 3: Therein, the image processing path for the VIS image E is identified on the left side, while the right arm shows the spectral image processing path in which the first image regions 4 are identified in automated fashion by means of the first selection step A and within the same path the second image regions 5 are identified in automated fashion with the aid of the second selection step B. The original spectral image G (recorded by the upper image sensor 18 in FIG. 2), to which the two selection steps A and B are applied, in this case comprises spectral information that deviates from the spectral information captured in the VIS image E. This is because while the VIS image E illustrates visible wavelengths, the image information of the spectral image G/H/I comprises fluorescence wavelengths. For example, these can be located outside of the visible wavelength range.

For example, if the first selection step A in FIG. 3 is realized with the aid of autofluorescence imaging, then the first image regions 4 can show adipose and nerve tissue, while further tissue types such as arteries, veins, muscles, organs such as the liver or metallic objects, which were likewise still visible in the image G, have already been filtered out in step A, as indicated (schematically) in image H. The second selection step B can then be implemented for example on the basis of diffuse reflectance spectroscopy (DRS) such that then the image I and the subsequent fused image F contain the position of the nerves as the desired image information to be determined. The further method step C thus represents image fusion between the image I and the image E. As a result, the image F thus shows an overlay image, consisting of the VIS image E and (as overlay) the desired tissue information 6 with regards to the precise position of the nerves in the operating region 9.

FIG. 4 shows a further possibility of how a method according to the invention can be used with an image recording system 1. This system 1 differs from that in FIG. 1 to the extent that the light source 15 is modulated over time, with the result that different wavelengths are transmitted as illumination light 11 and/or as excitation light 10 to the observed region 9 at different times.

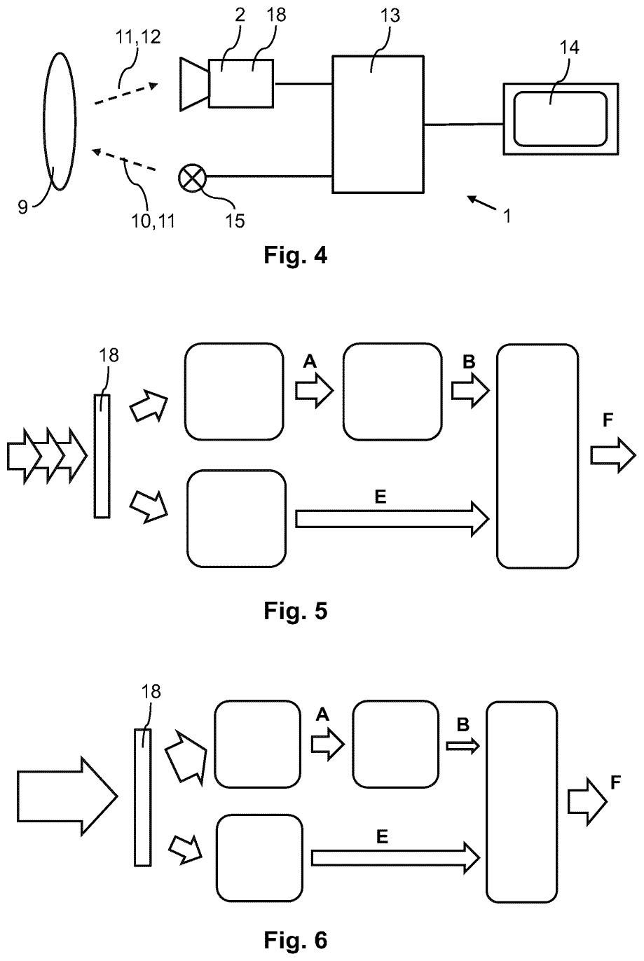

As shown in FIG. 5, a (temporally) sequential image capture using only a single image sensor 18 can therefore be implemented using the system 1 in FIG. 4, wherein the spectral information of the respective image 3 then differs, with the images 3 all being recorded by the image sensor 18. If this spectral information thus collected over relatively long periods of time is fused by way of appropriate image processing, then it is possible—as depicted in the lower image processing path in FIG. 5—to generate a VIS image E even though only a certain proportion of the visible wavelength range is available for imaging purposes at any specific time. By contrast, in the upper image processing path of FIG. 5, partial spectral information can be used at a specific time to carry out the above-explained selection steps A and B and thus determine the position of the target tissue type 6 within the image 3. In other words, the first selection step A and the second selection step B are therefore performed in the example of FIGS. 4 and 5 on the basis of partial spectral information temporally separated from overall spectral information of the at least one image 3.

FIG. 6 finally shows a further possible example of an implementation of a method according to the invention, with a hyperspectral image sensor 18 which is able to also record invisible wavelengths (for instance in the UV and/or in the IR wavelength range) in addition to visible wavelengths being used in this case. To this end, the HSI image sensor 18 can for example comprise appropriate spectral filters at the pixel level. The overall spectral information optically captured by the endoscope 2 and thus reaching the HIS image sensor 18 of FIG. 6 is thus again split spatially in this case, specifically on the basis of individual pixels of the image sensor 18. This then also allows the selection steps A and B to be carried out in the upper image processing path of FIG. 6 on the basis of partial spectral information, while spectral information deviating therefrom is used for imaging in the lower image processing chain of FIG. 6 in order to create the image E, which can be a VIS image in particular.

In FIG. 6, the thickness of the respective block arrow should serve here as indicator for the width of the respectively used spectral range, i.e. the wider the arrow, the more spectral information is processed in the respective image processing path. Hence, it is possible to identify that there is also a reduction in the spectral information in the first selection step A, and so the second selection step B can be applied to an (in comparison with the first selection step A) smaller spectral range. This makes it possible to utilize very much simpler optical measurement or imaging methods and image processing algorithms in the second selection step B; this is very advantageous, especially in endoscopic applications, when only a limited spectral resolution can be often obtained because no spectrometer is typically available. Moreover, this means substantially less information must be processed, and this facilitates the application of the method in real time, for instance in the case of a live video frame data stream.

In summary, a novel technical approach allowing the automated localization, within a recorded image 3, of a specific tissue type 6 within an operating region 9 using an image recording system 1 is proposed. To this end, provision is made for two temporally successive selection steps A and B to be used to identify and select those image regions 4, 5 within the image 3 which show the desired tissue type 6. In this case, the scope of the first selection step A includes both a spatial restriction of the image regions 5 and optionally a reduction in the spectral information to be processed and/or in the pixels to be processed of the respective image 3. Only in the second selection step B is the target tissue type 6 actually to be determined then finally identified within the image 3 and selected as second image regions 4 so that a user can ultimately locate this tissue type 6 within the region 9 observed using the image recording system 1 (cf. FIG. 2).

### LIST OF REFERENCE SIGNS

1 Image recording system
2 Endoscope
3 Image (recorded using 2/1, in particular live image as part of a video frame data stream)
4 First image region (identified within 3 in automated fashion)
5 Second image region (identified within 4 in automated fashion)
6 Target tissue type (=primary tissue type)
7 Secondary tissue type (belonging to the same tissue group as 6, but differing from 6)
8 Tertiary tissue type (deviating from 6 and 7; does not belong to the same tissue group as 6)
9 Region (in particular operating region, is observed by 1/2)
10 Excitation light (in particular non-visible, e.g. in UV; for exciting light emission in 9)
11 Illumination light (for allowing conventional imaging with visible wavelengths)
12 Fluorescence light
13 Controller, designed in particular as camera control unit
14 Monitor
15 Light source (multispectral or varying spectrally in time)
16 Overlay
17 Beam splitter (optionally with suitable spectral filters)
18 Image sensor

1. A method for automated localization of a target tissue type (6) within at least one image (3) recorded by an image recording system (1), the method comprising:
identifying and selecting at least one first image region (4) in automated fashion in a first selection step by a first imaging or measurement method within the at least one image (3), the at least one first image region (4) also visualizing at least one other secondary tissue type (7) that deviates from the target tissue type (6) in addition to the target tissue type (6), and
identifying and selecting at least one second image region (5) within the at least one already preselected first image region (4) in a second selection step by a second imaging or measurement method that deviates from the first imaging or measurement method, the at least one second image region (5) predominantly to exclusively visualizing the target tissue type (6) to be determined.

2. The method as claimed in claim 1, wherein at least one of the first selection step or the second selection step is performed in automated fashion by the image recording system (1) based on an image evaluation of the at least one image (3).

3. The method as claimed in claim 1, further comprising carrying out the first and second selection steps continually in real time, and hi-lighting the target tissue type (6) visually in a live video frame data stream.

4. The method as claimed in claim 3, wherein the hi-lighting allows a user to localize the target tissue type (6) within a region observed using the image recording system (1).

5. The method as claimed in claim 1, further comprising carrying out at least one of the first selection step or the second selection step based on partial spectral information which is spatially or temporally separated from overall spectral information relating to the at least one image (3).

6. The method as claimed in claim 1, wherein the first selection step reduces at least one of a quantity of image information or a quantity of spectral information processed in the second selection step.

7. The method as claimed in claim 1, the second selection step further comprises calculating color ratios based on at least two different color channels of the image recording system (1) for different picture elements in the at least one image (3).

8. The method as claimed in claim 7, wherein respective spectral ranges on the basis of which the color ratios are determined have a minimum width of at least 10 nm.

9. The method as claimed in claim 1, wherein the target tissue type (6) is nerve tissue.

10. The method as claimed in claim 9, wherein the at least first image region (4) also visualizes adipose tissue in addition to the nerve tissue.

11. The method as claimed in claim 1, wherein the first imaging or measurement method is selected from one of the following methods:
diffuse reflectance spectroscopy (DRS);
autofluorescence imaging, with a region (9) observed using the image recording system (1) being illuminated with excitation light (10);
fluorescence imaging, with a region (9) observed using the image recording system (1) being illuminated with excitation light (10);
laser speckle imaging (LSI), with a region (9) observed using the image recording system (1) being illuminated with coherent light;
imaging based on a polarization analysis with at least one of a region (9) observed using the image recording system (1) being illuminated with polarized light or the at least one image (3) being recorded with the aid of a polarization filter.

12. The method as claimed in claim 1, wherein the second imaging or measurement method is selected from one of the following methods:
diffuse reflectance spectroscopy (DRS) with a distinction being made between nerve tissue and adipose tissue based on diffuse reflectance spectroscopy such that the at least one second image region (5) predominantly to exclusively contains/visualizes nerve tissue;
fluorescence imaging using at least one fluorophore which is introduced into a region observed using the image recording system;
laser speckle imaging (LSI) with a region (9) observed using the image recording system (1) being illuminated with coherent light to this end;
imaging based on a polarization analysis with at least one of a region (9) observed using the image recording system (1) being illuminated with polarized light or the at least one image (3) being recorded with the aid of a polarization filter.

13. The method as claimed in claim 1, wherein the second selection step includes the use of
a fluorescence signal which spectrally deviates from a fluorescence signal used in the first selection step, or
an optically captured movement signal, or
optically captured information regarding a structure of the tissue for the further differentiation between tissue in the second selection step.

14. The method as claimed in claim 1, wherein the at least one first image region (4) is computer ascertained using artificial intelligence trained using measurement data measured for different tissue types using the first imaging method and an image recording system (1).

15. The method as claimed in claim 1, further comprising carrying out the method during an endoscopic procedure performed with an endoscope (2) as part of the image recording system (1).

16. A medical image recording system (1), comprising:
a controller (13) configured to conduct the method as claimed in claim 1 based on image data recorded using the image recording system (1).

17. The medical image recording system (1), wherein the medical image recording system (1) comprises an endoscope (2), a microscope or a macroscope.
